(19) United States
(12) Patent Application Publication
(10) Pub. No.: US 2005/0040026 A1
(43) Pub. Date: Feb. 24, 2005

(54) Process for the process management of an extractive distillation plant, process control system and extractive distillation plant

(76) Inventors: Joachim-Walter Grub, Dormagen (DE); Joerg-Uwe Michel, Salierstrasse (DE); Peter Adolf Helmut Prinz, Dormagen (DE); Franz Ferdinand Rhiel, Dormagen (DE); Markus Vetter, Muhlheim (DE); Horst Werner Eugen Willi Wegner, Dormagen (DE)

Correspondence Address: Finnegan Henderson Farabow;Garrett & Dunner, 1300 I Street NW, Washington, DC, 20005, US

(21) Appl. No.: 10/488,473

(22) PCT Filed: Aug. 28, 2002
(86) PCT No.: PCT/IB02/03888

(30) Foreign Application Priority Data
Sep. 4, 2001 (DE) 101 44 239.4

(51) Int. Cl. B01D 3/42; B01D 3/34; C07C 7/00

(52) U.S. Cl. 203/1; 203/3; 203/2; 203/58; 203/DIG. 18; 585/804; 585/865

(57) ABSTRACT

A process for the process management of an extractive distillation plant for the separation of pure aromatics from a parent mixture of aromatic and non-aromatic hydrocarbons with the use of a selective auxiliary substance is described, in which the non-aromatic hydrocarbons are obtained as raffinate and the minimum energy input is set in accordance with an optimisation goal definable by controlled variables, in which the energy input is further set in accordance with the current raffinate quantity in combination with an online measurement of the non-aromatics quantity in the parent mixture. The process according to the invention permits a systematic and fully automatic setting of the plant limits.

The present invention relates to a process for the process management of an extractive distillation plant for the separation of pure aromatics from a parent mixture of aromatic and non-aromatic or aliphatic hydrocarbons with the use of a selective auxiliary substance, together with a process control system, an extractive distillation plant and a computer program for carrying out such a process.

Extractive distillation processes are known and are used for the extractive separation of mixtures with close-boiling components whose separation is possible otherwise only with an uneconomic number of separation stages and high energy input. With extractive distillation the separation factor for components that are to be separated from a parent mixture is increased by addition of an auxiliary substance, in general of a selectively acting solvent or solvent mixture. The improved separation is achieved by the fact that the auxiliary substance possesses a higher affinity with one or more components of the parent mixture, whereby their vapour pressures are significantly changed, so that a separation by distillation is possible.

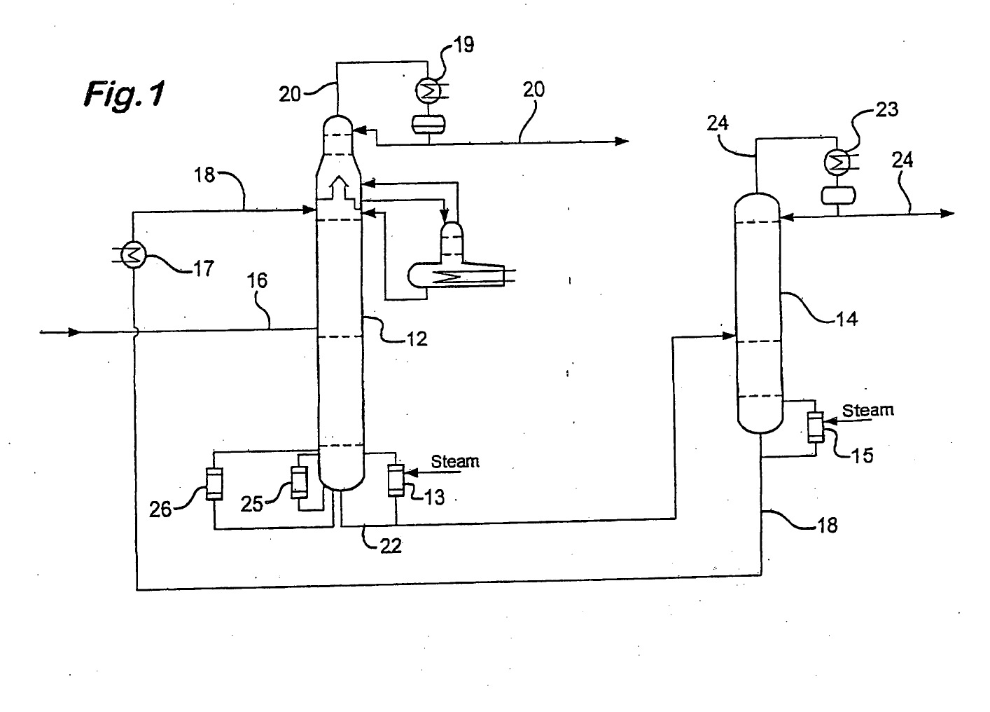

An extractive distillation process of this kind is shown diagrammatically in FIG. 1. The extractive distillation process is carried out in two distillation columns 12, 14. The parent mixture 16 is introduced into the central part and the selective auxiliary substance 18 into the upper part of the extractive distillation column 12. In the extractive distillation column 12 the lower-boiling components are drawn off out of the parent mixture 16 overhead as raffinate 20, while the target product, namely the higher-boiling components, is collected together with the auxiliary substance as bottom product 22 of the distillation. The bottom product 22 is fed out of the extractive distillation column 12 into the centre of a downstream stripper column 14, in which the auxiliary substance and the target product from the parent mixture are separated from one another by distillation. The target product is drawn off overhead at 24. The auxiliary substance collecting in the bottom 11 of the stripper column 14 is returned into the extractive distillation column 12 via at least one cooler 17. The energy content of the auxiliary substance may be used to heat the bottom of the extractive distillation column 12 by being pumped via the closed-circuit heater 25. In addition, the heating both of the bottom of the extractive distillation column and the bottom of the stripper column 14 takes place by means of steam feeding via closed-circuit heaters 13 and 15. The bottom of the extractive distillation column is in addition heated with condensate from other plant sections (26).

Extractive distillation processes of this kind are used today in a multiplicity of large-scale processes, for example to obtain pure aromatics or butadiene and to separate butanes and butenes. Extractive distillation processes are in addition used to separate aromatics from petrochemical products.

The following have proved to be particularly effective auxiliary substances: selectively acting solvents such as, for example, N-substituted morpholines, in particular N-formyl morpholine, N-methyl pyrrolidone and dimethyl formamide.

It is in addition known from EP 0 418 622 A2 that the required amount of a selective solvent and the temperature of the solvent during feeding into the extraction distillation column are directly correlated to one another.

The quality of the extractive distillation depends on various operating parameters, such as e.g. pressure and temperature. In order to achieve an optimum separation efficiency and yield with minimum supply of energy to the extractive distillation column, a continuous adjustment of the operating parameters to the current conditions is required.

Controlled variables which determine the set values of the separation efficiency may be e.g. the aromatics content or the auxiliary substance content in the raffinate or the non-aromatics content in the pure aromatics.

Disturbance variables which cause fluctuations in the operation of a column may be e.g. a changing composition and temperature of the parent mixture or auxiliary substance fed to the extractive distillation column or a changing ambient temperature. Disturbances of this kind require immediate intervention in the operating parameters, e.g. intervention in the energy supply, in order to be able to meet the desired product specification and yield.

There is therefore a need for processes by means of which the operation of an extractive distillation column may be optimised with simultaneous minimisation of the energy requirement.

The optimisation of the column operation may take place e.g. by means of equipment measures. Thus it is known from U.S. Pat. No. 5,215,629 and U.S. Pat. No. 5,252,200 to pre-heat the parent mixture in the feed to the extractive distillation column by indirect heat exchange with the selective auxiliary substance. In addition, it is known from U.S. Pat. No. 6,007,707 to arrange above the auxiliary substance feed to the extractive distillation column a further auxiliary substance feed, by means of which approx 0.5 to 10% more of the total amount of auxiliary substance passes into the extractive distillation column. A reduction of the aromatics content in the raffinate is achieved in this way.

The optimisation of the column operation may also be performed by suitable process control, in particular by online process optimisation. The online process optimisation makes use for the control of the column operation of measured values determined online (e.g. temperature, pressure, concentrations, ambient conditions, for example the ambient temperature). The column is thereby no longer run constantly in a predetermined operating state, but optimised in terms of the framework conditions applying at the time, such as raw material costs and achievable product prices.

The so-called feedforward strategy has particular importance here. In the feedforward strategy changes in parameters are included before they exert an influence on the column. By means of the feedforward strategy, it is possible, by the evaluation of recorded measured values, for the requisite operating state of the distillation column to be set with the change in advance. An example of such a parameter is e.g. a changed composition of the parent mixture, which leads with constant energy input to a disturbance of the column equilibrium.

It is known from U.S. Pat. No. 4,488,936 for the energy supply to an extractive distillation column to be set by means of a temperature measurement, a temperature difference measurement in the upper part of the extractive distillation column, or the analysis result of a gas chromatographic provided in the vicinity of the column head, in such a way that the extract content in the raffinate is as low as possible.

WO 9 829 787 A1 discloses an online monitoring and setting of the operating parameters in order to control the product properties in the manufacture of butyl rubber. The measured values are recorded in situ with a spectrometer. The anticipated product properties are then calculated on the basis of relationships stored in the process control system. The difference between predicted and desired product quality serves as a controlled variable for the control of the operating parameters. Such a form of process control may be used in a large number of different chemical plants.

Hydrocarbon Processing, June 1989, pp. 64 to 71, discloses an online process management for catalytic cracking processes based on specified optimisation goals, in which-in particular feed rates in reactors and columns are used in accordance with a feedforward strategy.

WO 0 020 939 A1 discloses the use of neuronal networks for process management. Process states are here predicted by means of neuronal networks. Said items of information may be used for the control of the process.

Europa Chemie 18/99 discloses that online process management concepts of this kind are used successfully also in extractive distillation.

In Erdöl und Kohle 47/9, 1994 an online system for the optimum operational management of an extractive distillation with the solvent N-formyl morpholine is described. The quality indices aromatics yield, product purity and energy consumption, which are critical for the plant operation, are used as a basis for the optimisation. Taking into account further important conditions, a profit function was drawn up, which is stored in the process control system of the plant. There is generated from the process simulation with the current process data a set of controller setpoint values which corresponds to an optimum operation of the distillation column under the stated conditions. The plant personnel may then apply the controller setpoint values to the process control system.

Conversely the present invention is based on the object of providing a process for the fully automatic process management of an extractive distillation plant with an improved profitability.

To achieve the object, a process for the process management of an extractive distillation plant with the features of claim 1 is proposed. In addition, a process control system with the features of claim 15, an extractive distillation plant with the features of claim 16 and a computer program with the features of claim 17 are proposed.

Consequently the minimum energy input in accordance with an optimisation target definable by controlled variables is automatically set in the process according to the invention. According to the invention the energy input is in addition set in accordance with the current raffinate quantity in combination with an on-line measurement of the non-aromatics quantity in the parent mixture.

There is meant by the current raffinate quantity the quantity of raffinate that leaves the extractive distillation column. It is determined from a quantitative measurement after the raffinate tank of the extractive distillation column and a measurement of the raffinate tank level change. The current raffinate quantity is used in combination with the quantity of non-aromatics in the parent mixture to preserve the mass balance of the non-aromatics. Since all changes in the energy balance of the column have a direct influence on the current raffinate quantity as a result of quantity and temperature fluctuations of the mass flows entering the column, and also of changes in the ambient conditions, e.g. precipitation and ambient temperature, the delay in time which occurs because of the buffer effect of the raffinate tank and dead times caused by plant and measurement procedures is offset by the determination and incorporation of the current raffinate quantity. A more rapid allowance for changes in the state of the extractive distillation is therefore possible by determination of the current raffinate quantity. The process according to the invention therefore permits a systematic setting of the plant limits.

There are provided as optimisation goals according to the invention in particular a maximisation of the throughput through the extractive column, an exact observance of a predetermined purity of the pure aromatics to be obtained, a maximisation of the purity of the raffinate and/or a minimisation of the loss, or maximisation of the yield, of pure, aromatics.

There serve as controlled variables in particular the non-aromatics content in the pure aromatics, which is predetermined as a product specification, and/or the aromatics content in the raffinate, with which the yield is set.

The minimum auxiliary substance feed quantity is further set as an advantageous development of the invention. The quantity of auxiliary substance required for the separation is obtained from an online measurement of the composition and the quantity of the parent mixture in the extractive distillation column. The required auxiliary substance feed quantity may further be dependent on the feed temperature of the parent mixture and be set accordingly according to the invention.

For the optimisation of the operation of the extractive distillation plant the auxiliary substance feed quantity is advisedly co-ordinated with the energy input into the extractive distillation column and set in such a way that the non-aromatics specification of the pure aromatics is met, a high aromatics yield with minimum energy input is %achieved, and the plant capacity is fully exploited in terms of a: throughput maximisation.

In addition, with advantage a setting of the auxiliary substance feed quantity in accordance with an online measurement of the feed temperature of the auxiliary substance is provided, since the auxiliary substance quantity required for the,separation is dependent on the auxiliary substance feed temperature particularly when particular solvents or solvent mixtures are used as auxiliary substance. Such solvents are e.g. N-substituted morpholines, in particular N-formyl morpholine, N-methyl pyrrolidone (NMP), dimethyl formamide and similar. The selectivity of the solvent NMP for the separation of a parent mixture of benzene, methylcyclohexane and other non-aromatics by means of extractive distillation is, for example, inversely proportional to its feed temperature.

With advantage an auxiliary substance reference quantity, i.e. a temperature-compensated auxiliary substance quantity, is introduced as an auxiliary controlled variable which automatically adjusts the auxiliary substance feed quantity to the feed temperature, so that the separation efficiency is held constant. By adjustment of the auxiliary substance feed quantity on a change in the auxiliary substance feed temperature, the energy balance of the column is disturbed considerably less than is the case with a control of the auxiliary substance feed quantity without allowance for the auxiliary substance feed temperature.

In particular it may also be provided that, in order to overcome dead times occurring in plant and sensors, changes in feed temperature and feed quantity of the auxiliary substance entering the extractive distillation column may be switched directly onto the energy input as feedforward.

With the process according to the invention all of the above-mentioned relationships of the measured variables are determined with advantage from measurements and simulations and stored in a process control system in the form of models based on physical laws. The input measured variables, e.g. feed quantity, temperature and composition of the parent mixture, may therefore be used in accordance with a feedforward strategy and implemented jointly with a feedback strategy on the basis of the controlled variables. A combination of feedforward and feedback strategies therefore makes it possible to also use, for the determination of measured values, measuring devices that supply discontinuous measured values and exhibit dead times of, for example, 20 minutes.

The feedback controls are based on measured values e.g. from measurement of the non-aromatics quantity in the parent mixture or of the non-aromatics content in the pure aromatics. Said measured variables are with advantage clearable separately. On the occurrence of a fault in an analytical or measuring device said measured values may therefore be removed from the control circuit, so that the remaining components of the feedback control may continue to be used.

As a further advantageous development of the invention the measurement of the non-aromatics content in the pure aromatics and/or of the aromatics content in the raffinate is carried out in each case by means of an analytical device which measures the concentration in the product stream leaving the respective distillate or raffinate tank. In particular it is provided according to the invention to measure the controlled variables by means of chromatographs which are connected into closed control loops. A manual forwarding of control setpoints is therefore not necessary. The plant operator simply stipulates the required product specification and the desired yield. The setting and changing of setpoints for all further controllers then take place automatically. This also applies to the case of a change of the optimisation goal, e.g. the maximisation of the throughput at the cost of lower yield. The system thus sets automatically the minimum energy input for the respective optimisation strategy.

As a particularly advantageous development of the process according to the invention a continuous checking of the output data of chromatographs and other analytical devices for plausibility and consistency on the basis of other process variables and physical laws is provided. Thus dropouts and outliers are identified among the output signals of the analytical devices and may be removed from the control circuit.

The process according to the invention is in particular suitable for the obtaining of pure benzene from the benzene cut of a petroleum fraction with the use of N-methyl pyrrolidone (NMP) as selective auxiliary substance. In this case the special feature lies in the fact that the selectivity of the auxiliary substance rises with falling inlet temperature into the column.

Further advantages and developments of the invention follow from the description and the attached drawing.

It goes without saying that the features given above and those still to-be explained below are usable not only in the respective combination given, but also in other combinations or on their own, without exceeding the scope of the present invention.

The invention is represented diagrammatically by means of an embodiment in the drawing and will be described in detail below with reference to the drawing.

FIG. 1 shows an extractive distillation from the prior art.

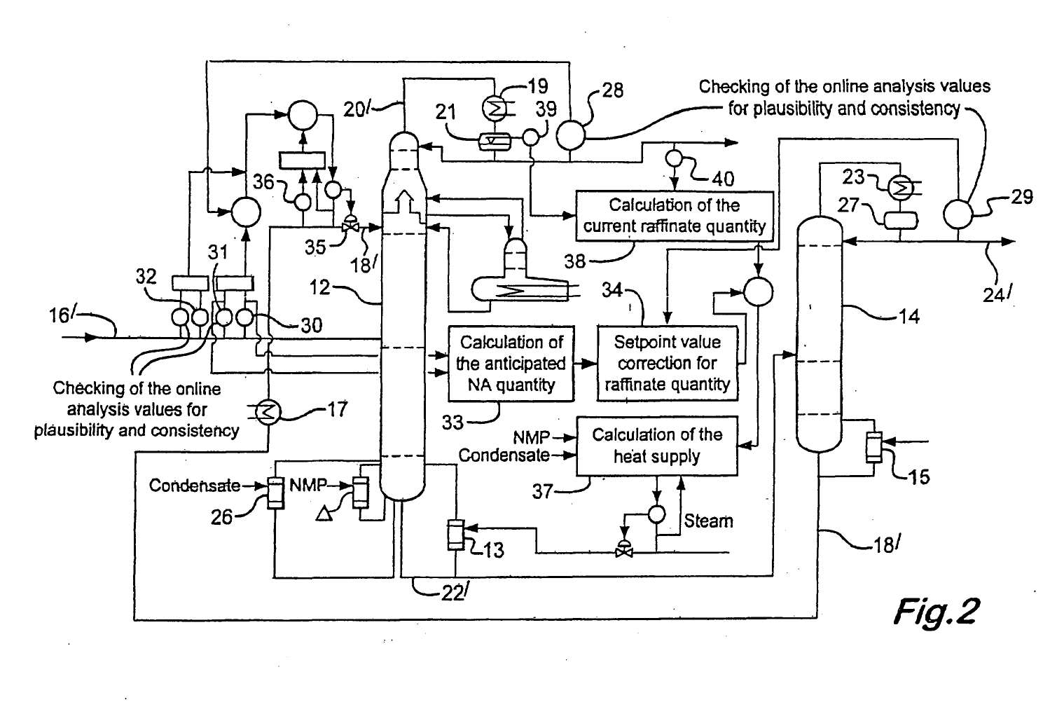

FIG. 2 shows an extractive distillation process which is managed by a process according to the invention.

The process according to the invention for the process management of an extractive distillation plant is explained in detail below from the example of extractive distillation by the Distapex process, in which pure benzene is obtained from the benzene cut of a petroleum fraction using N-methyl pyrrolidone (NMP) as selective auxiliary substance. In the embodiment described below the optimisation goal consists in the observance of the product specification for pure benzene and a high pure benzene yield with low energy input together with full use of the plant capacity by maximisation of the throughput

As shown in FIG. 2, the benzene cut is introduced as parent mixture 16′ into the central part and NMP as selective auxiliary substance 18′ into the upper part of the extractive distillation column 12. The benzene cut 16′ contains inter alia benzene, methylcyclohexane and other non-aromatics in an approximate composition of 70 to 90% benzene, 0.1 to 0.5% methylcyclohexane and 10 to 30% other non-aromatics. In the extractive distillation column 12 the non-aromatics are drawn off overhead as raffinate 20′, while benzene together with the NMP is obtained as bottom product 22′. The bottom product 22′ is fed out of the extractive distillation column 12 into a downstream stripper column 14, in which NMP and pure benzene are separated by distillation, wherein the NMP obtained in the bottom 11′ of the stripper column 14 is returned into the extractive distillation column 12.

In order to determine the anticipated non-aromatics quantity (33), the feed quantity of the benzene cut is measured online at 30 and the aromatics content of the benzene cut at 31. The analysis value for the aromatics content of the benzene cut at 31 is also checked for plausibility and consistency. The non-aromatics quantity in the benzene cut is utilised in the feedback control as a basis for determining the quantity of raffinate to be vaporised and hence for determining the requisite energy input into the extractive distillation column in accordance with a feedforward strategy. In order to offset measuring inaccuracies and to take account of the aromatics quantity contained in the raffinate 20′, a setpoint correction term is at 34 added to the non-aromatics quantity in the parent mixture. The setpoint value for the raffinate to be vaporised is obtained from the sum. The setpoint correction term serves in addition as a manipulated variable for meeting the non-aromatics specification in the pure benzene 24′.

The energy input, which is required for the separation efficiency of the extractive distillation column 12, is supplied via closed-circuit heaters 25, 26 and 13 by heat exchange with energy sources. In the embodiment of FIG. 2 the energy feed to the extractive distillation column 12 takes place via three energy sources., namely at 25 through the hot NMP substance stream from the stripper column 14, at 26 through hot condensate from other plant sections and at 13 through water vapour. The amount of energy supplied must be such that the non-aromatics contained in the benzene cut 16′ leave the column as raffinate 20′ overhead. The supply of steam required is calculated at 37 in accordance with the fluctuations in the energy supply supplied by the other energy sources and thus serves as a manipulated variable for meeting the non-aromatics specification in the pure benzene 24′.

In order to determine the non-aromatics content (31) and the feed quantity (30) of the benzene cut, in addition to the feedback circuit the energy supply also forms a basis for the control of the quantity of NMP fed into the extractive distillation column 12 in accordance with a feedforward strategy at 35. In particular when a benzene cut is used as parent mixture, allowance for the methylcyclohexane content of the benzene cut is of importance. The NMP feed quantity required is also controlled as a function of an online measurement of the feed temperature of the benzene cut at 32 and an online measurement of the feed temperature of the NMP at 36. By adjustment of the NIP feed quantity with a change in the NMP feed temperature, the energy balance of the column is in this embodiment disturbed by less than 75% than is the case with a feedback control of the NMP quantity, in which the NMP feed temperature is not taken into account.

In the extractive distillation column 12 the NMP preferably undergoes by virtue of its chemical structure interactions with the aromatic benzene and thus lowers the latter's vapour pressure. The bottom product 22′ consists of benzene and NMP and is passed into the central area of a solvent stripper column 14. There, pure-benzene 24′ is separated overhead by distillation, condensed via a cooler 23 and obtained as distillate. The non-aromatics content of the pure benzene 24′ is determined in the pure benzene stream 24′ by means of a gas chromatograph 29 arranged downstream of the distillate tank 27, and after checking of the analysis values for plausibility and consistency, used for the setpoint correction for the raffinate quantity (34) and hence for calculating the energy input into the extractive distillation column (37).

The energy feed to the stripper column 14 takes place likewise via a closed-circuit heater 15 which is fed by a suitable energy source, e.g. water vapour. The NMP is drawn off at the bottom 11′ of the stripper column 14 and passed once again into the upper part of the extractive distillation column 12. In addition, the energy content of the NMP from the bottom of the stripper column 14 is used to heat the extractive distillation column 12 by means of heat exchanger 25.

The non-aromatics and traces of the NMP are obtained at the head of the extractive distillation column 12 as raffinate 20′, condensed via a cooler 19 and collected in a raffinate tank 21. The aromatics content of the raffinate 20′ is determined in the raffinate stream by means of a gas chromatograph 28 arranged downstream of the raffinate tank 21 and, after checking of the analysis values for plausibility and consistency, used, on the basis of the setpoint value preset as controlled variable, for setting the quantity of NMP fed into the distillation column in accordance with a feedback strategy.

The current raffinate quantity is determined at 38 from a measurement (39) of the tank level of the raffinate tank 21 and a raffinate quantity measurement (40) downstream of the raffinate tank 21. The current raffinate quantity is used to meet the mass balance of the non-aromatics and controls on said basis the energy input into the extractive distillation column 12, i.e. the amount of steam supplied, in accordance with a feedforward strategy, in order to meet the specification of the non-aromatics in the benzene with minimum energy consumption. The energy input into the extractive distillation column 12 is set in such a way that the non-aromatics contained in the benzene cut 16′ fully leave the column as raffinate 20′ overhead. The meeting of the non-aromatics mass balance is therefore a pre-condition for the meeting of the non-aromatics specification set as controlled variable in the pure aromatics.

### EXAMPLE

With a typical operating state of the extractive distillation plant the NMP quantity must, in order to preserve the separation efficiency with a lowering of the NMP inlet temperature by 1 K, be reduced by 0.9 t/h. At the same time a working point-dependent additional steam quantity of approx. 50 kg/h is required.

By means of the process for process management according to the invention the non-aromatics content in the pure benzene is held within a fluctuation range of ±20-ppm. With the previous method of operation said fluctuation range came to ±60 ppm. Due to the better control quality, therefore, the setpoint value for the product purity is set 40 ppm closer to the specification limit.

Plant operation close to the specification limit and the consistent use of the lowest possible NMP feed temperature permit a better setting of the plant limits and hence an increase in capacity of more than 3%.

The process for process management according to the invention thus permits the deliberate use of temperature fluctuations (day-night, summer-winter) in order to achieve an optimisation goal such as a throughput maximisation by permanent setting of the plant limits. In addition, the process according to the invention ensures fully automatic operation with load variations, and also with abrupt changes in external conditions such as violent showers of rain suddenly occurring.

1. Process for the process management of an extractive distillation plant for the separation of pure aromatics from a parent mixture of aromatic and non-aromatic hydrocarbons with the use of a selective auxiliary substance, in which the non-aromatic hydrocarbons are obtained as raffinate and the minimum energy input is set in accordance with an optimisation goal definable by controlled variables, characterised in that the energy input is further set in accordance with the current raffinate quantity in combination with an online measurement of the non-aromatics quantity in the parent mixture.

2. Process according to claim 1, characterised in that the optimisation goal is a maximisation of the throughput, a meeting of the purity of the pure aromatics, a maximisation of the purity of the raffinate and/or a minimisation of the loss of pure aromatics.

3. Process according to claim 1 or 2, characterised in that the controlled variable is the non-aromatics content in the pure aromatics and/or the aromatics content in the raffinate.

4. Process according to one of the previous claims, characterised in that the auxiliary substance feed quantity is set.

5. Process according to one of the previous claims, characterised in that the auxiliary substance feed quantity is set as a function of an online measurement of the feed quantity, the composition and the temperature of the parent mixture respectively.

6. Process according to claim 4 or 5, characterised in that the auxiliary substance feed quantity is set as a function of an online measurement of the auxiliary substance temperature on entrance into the extractive distillation column.

7. Process according to one of the previous claims, characterised in that the auxiliary substance is a selectively acting solvent or a selectively acting solvent mixture.

8. Process according to one of the previous claims, characterised in that the auxiliary substance is selected from N-substituted morpholines, in particular N-formyl morpholine, N-methyl pyrrolidone, dimethyl formamide and mixtures thereof

9. Process according to one of the previous claims, characterised in that a measurement of the non-aromatics content in the pure aromatics and/or of the aromatics content in the raffinate takes place by means of an analytical device from the product stream leaving distillate or raffinate tanks.

10. Process according to claim 9, characterised in that the analytical devices are chromatographs connected into the control loop.

11. Process according to one of the previous claims, characterised in that the relationships of the measured variables are stored in a process control system in the forms of models based on physical laws.

12. Process according to one of the previous claims, characterised in that each measured variable is clearable separately.

13. Process according to claim 9 or 10, characterised in that the output signals are checked for plausibility and consistency by analytical devices on the basis of rigorous models.

14. Process according to one of the previous claims, characterised in that pure benzene is separated from the benzene cut of a petroleum fraction with the use of N-methyl pyrrolidone as selective auxiliary substance.

15. Process control system for carrying out a process according to one of claims 1 to 14.

16. Extractive distillation plant with a process control system according to claim 15.

17. Computer program with program code which, when the computer program is run in a computer of a process control system, is capable of executing a process according to one of claims 1 to 14.

18. Computer program according to claim 17, which is stored on a computer-readable medium.
